(19) United States
(12) Patent Application Publication
(10) Pub. No.: US 2024/0342247 A1
(43) Pub. Date: Oct. 17, 2024

(54) COMPOSITIONS AND METHODS FOR ACTIVATING A NEURAL RECEPTOR

(71) Applicant: GILA THERAPEUTICS, INC., Minneapolis, MN (US)

(72) Inventor: Thomas VASICEK, Minneapolis, MN (US)

(21) Appl. No.: 18/577,369

(22) PCT Filed: Jul. 8, 2022
(86) PCT No.: PCT/US2022/036437

§ 371 Date: Jan. 8, 2024

Related U.S. Application Data
(60) Provisional application No. 63/219,409, filed on Jul. 8, 2021.

(51) Int. Cl. A61K 38/22; A61K 38/23; A61K 38/26; A61K 38/28

(52) U.S. Cl. CPC A61K 38/22; A61K 38/2264; A61K 38/23; A61K 38/26; A61K 38/28

(57) ABSTRACT

The invention provides compositions and methods for inducing activating a neural receptor. The compositions include a metabolic hormone, which can be used to provide treatment for an addiction or mood disorder in a subject.

### BACKGROUND OF THE INVENTION

The prevalence of addiction and mood disorders continues to increase worldwide. However, there is still a lack of effective, long-term, non-invasive treatments for addiction and mood disorders. Treatment with existing therapeutics often does not produce suitable therapeutic effects. Accordingly, new treatments are needed.

### SUMMARY OF THE INVENTION

In one aspect, the invention features a method of activating a neural receptor in a subject by administering to the subject a composition that includes an agent selected from Peptide YY (PYY), glucagon-like peptide 1 (GLP-1), leptin, amylin, insulin, and calcitonin, or an analog, variant, or biologically active fragment thereof. The composition activates the neural receptor without substantially changing the concentration of the agent in the blood of the subject.

In some embodiments, activation of the neural receptor provides treatment for an addiction or a mood disorder in the subject in need thereof.

In some embodiments, activation of the neural receptor provides treatment for an addiction.

In some embodiments, the addiction includes a craving or a dependency for alcohol, cocaine, opioids, nicotine, heroin, marijuana, 3,4-methylenedioxy-methamphetamine, caffeine, mescaline, methamphetamine, amphetamine derivatives, dextromethorphan, loperamide, phenylcyclohexyl piperidine, stimulants, steroids, cannabinoids, cathinones, lysergic acid diethylamide, gambling, sex, inhalants, sedatives, hypnotics, tobacco, anxiolytics, hallucinogens, food, or a combination thereof.

In some embodiments, activation of the neural receptor provides treatment for a mood disorder.

In some embodiments, the mood disorder includes depression, anxiety, dysthymia, bipolar disorder, a medication-induced mood disorder, obsessive compulsive disorder, binge eating disorder, or a substance-induced mood disorder.

In some embodiments, the composition is administered topical-lingually (e.g., topically to the lingual epithelium). The composition may be formulated, e.g., as an oral dissolving tablet, a lozenge, a film, a spray, a semisolid, a particulate, or in a lipid-based carrier.

In some embodiments, the composition is administered intranasally. The composition may be formulated, e.g., as a spray, a semisolid, a particulate, or in a lipid-based carrier.

In some embodiments, the addiction or mood disorder does not include of post-traumatic stress disorder (PTSD), traumatic brain injury (TBI) Alzheimer's disease, memory loss, cognitive defects, stress, stroke, or a neurodegenerative disorder.

In some embodiments, the method does not include administration of insulin.

In some embodiments, the method does not include intranasal administration of insulin.

In some embodiments, the method does not include intranasal administration of insulin for the treatment of Alzheimer's disease.

In some embodiments, the composition is administered topically to the gastrointestinal (GI) tract. The composition may be formulated, e.g., as a GI patch.

In some embodiments, the composition is administered intrarectally. The composition may be formulated, e.g., as a suppository.

In some embodiments, the dose of the agent (e.g., PYY, GLP-1, leptin, amylin, insulin, or calcitonin, or an analog, variant, or biologically active fragment thereof) is from 1 ng to 20 mg per 100 kg body weight. For example, the dose of the agent may be from 1 ng to 10 ng per 100 kg body weight, e.g., 1 ng, 2 ng, 3 ng, 4 ng, 5 ng, 6 ng, 7 ng, 8 ng, 9 ng, or 10 ng per 100 kg body weight, e.g., from 10 ng to 100 ng per 100 kg body weight, e.g., 20 ng, 30 ng, 40 ng, 50 ng, 60 ng, 70 ng, 80 ng, 90 ng, or 100 ng per 100 kg body weight, e.g., from 100 ng to 1 μg per 100 kg body weight, e.g., 200 ng, 300 ng, 400 ng, 500 ng, 600 ng, 700 ng, 800 ng, 900 ng, or 1 μg per 100 kg body weight, e.g., from 1 μg to 10 μg per 100 kg, e.g., 2 μg, 3, μg, 4 μg, 5 μg, 6 μg, 7 μg, 8 μg, 9 μg, or 10 μg per 100 kg body weight, e.g., from 10 μg to 100 μg per 100 kg body weight, e.g., 10 μg, 20 μg, 30 μg, 40 μg, 50 μg, 60 μg, 70 μg, 80 μg, 90 μg, or 100 μg per kg body weight, e.g., from 100 μg to 1 mg per kg of body weight, e.g., 100 μg, 200 μg, 300 μg, 400 μg, 500 μg, 600 μg, 700 μg, 800 μg, 900 μg, or 1 mg per kg body weight, or e.g., from 1 mg to 20 mg per 100 kg body weight, e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg per 100 kg body weight.

In some embodiments, the composition includes PYY or a variant, analog, or biologically active fragment thereof. In some embodiments, the PYY fragment is PYY(3-36).

In some embodiments, the composition includes GLP-1 or a variant, analog, or biologically active fragment thereof.

In some embodiments, the composition includes leptin or a variant, analog, or biologically active fragment thereof.

In some embodiments, the composition includes amylin or a variant, analog or biologically active fragment thereof.

In some embodiments, the composition includes calcitonin or a variant, analog, or biologically active fragment thereof.

In some embodiments, the neural receptor is a Y receptor (YR), a Y1 receptor (Y1R), a Y2 receptor (Y2R), a Y4 receptor (Y4R), a Y5 receptor (Y5R), a G-protein coupled receptor (GPRCR), a leptin receptor (LEPR), a GLP-1 receptor (GLP-1R), an insulin receptor (INSR), a glucose-dependent insulinotropic polypeptide receptor (GIPR), a ghrelin receptor (GHS-R), a cholecystokinin A receptor (CCKAR), a cholecystokinin B receptor (CCKBR), or a calcitonin receptor (CALCR).

In another aspect, the invention features a composition that includes an agent selected from PYY, GLP-1, leptin, amylin, and calcitonin, or an analog, variant, or biologically active fragment thereof. The composition is formulated for intranasal administration and activates a neural receptor of a subject without substantially changing the concentration of the agent in the blood of the subject. The composition may be formulated, e.g., as a spray, a semisolid, a particulate, or in a lipid-based carrier.

In another aspect, the invention features a composition that includes an agent selected from PYY, GLP-1, leptin, amylin, insulin, and calcitonin, or an analog, variant, or biologically active fragment thereof. The composition is formulated for topical administration to the GI tract and activates a neural receptor of a subject without substantially changing the concentration of the agent in the blood of the subject. The composition may be formulated, e.g., as a GI patch.

In another aspect, the invention features a composition that includes an agent selected from PYY, GLP-1, leptin, amylin, insulin, and calcitonin, or an analog, variant, or biologically active fragment thereof. The composition is formulated for intrarectal administration and activates a neural receptor of a subject without substantially changing the concentration of the agent in the blood of the subject. The composition may be formulated, e.g., as a suppository.

In some aspect of any of the above compositions, the dose of the agent is from 0.1 ng to 20 mg. For example, the dose of the agent may be from 0.1 ng to 1 ng, e.g., 0.2 ng, 0.3 ng, 0.4 ng, 0.5 ng, 0.6 ng, 0.7 ng, 0.8 ng, 0.9 ng, or 1 ng, e.g., 1 ng to 10 ng, e.g., 1 ng, 2 ng, 3 ng, 4 ng, 5 ng, 6 ng, 7 ng, 8 ng, 9 ng, or 10 ng, e.g., from 10 ng to 100 ng, e.g., 20 ng, 30 ng, 40 ng, 50 ng, 60 ng, 70 ng, 80 ng, 90 ng, or 100 ng, e.g., from 100 ng to 1 μg, e.g., 200 ng, 300 ng, 400 ng, 500 ng, 600 ng, 700 ng, 800 ng, 900 ng, or 1 μg, e.g., from 1 μg to 10 μg per 100 kg, e.g., 2 μg, 3, μg, 4 μg, 5 μg, 6 μg, 7 μg, 8 μg, 9 μg, or 10 μg, e.g., from 10 μg to 100 μg, e.g., 10 μg, 20 μg, 30 μg, 40 μg, 50 μg, 60 μg, 70 μg, 80 μg, 90 μg, or 100 μg per kg body weight, e.g., from 100 μg to 1 mg, e.g., 100 μg, 200 μg, 300 μg, 400 μg, 500 μg, 600 μg, 700 μg, 800 μg, 900 μg, or 1 mg, e.g., from 1 mg to 20 mg, e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg.

In some embodiments, the composition includes PYY or a variant, analog, or biologically active fragment thereof. In some embodiments, the PYY fragment is PYY(3-36).

In some embodiments, the composition includes GLP-1 or a variant analog, or biologically active fragment thereof.

In some embodiments, the composition includes leptin or a variant, analog, or biologically active fragment thereof.

In some embodiments, the composition includes amylin or a variant, analog, or biologically active fragment thereof.

In some embodiments, the composition includes insulin or a variant, analog, or biologically active fragment thereof.

In some embodiments, the composition does not include insulin or an analog, variant, or biologically active fragment thereof.

In some embodiments, the composition includes calcitonin or a variant, analog, or biologically active fragment thereof.

In some embodiments, the neural receptor is a YR, a Y1R, a Y2R, a Y4R, a Y5R, a GPCR, a LEPR, a GLP-1R, an INSR, a GIPR, a GHS-R, a CCKAR, a CCKBR, or a CALCR.

### Definitions

To facilitate the understanding of this invention, a number of terms are defined below. Terms defined herein have meanings as commonly understood by a person of ordinary skill in the areas relevant to the invention. Terms such as “a,” “an,” and “the” are not intended to refer to only a singular entity but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, the term “subject,” refers to a human or non-human animal (e.g., a mammal).

As used herein, the terms “topical-lingual administration,” “topical-lingually,” or variations thereof, refer to the local administration of a metabolic hormone to the epithelium of the mouth and/or tongue of a subject with substantially no change in the levels of metabolic hormone in the blood of the subject (e.g., substantially no systemic exposure).

### DETAILED DESCRIPTION OF THE INVENTION

In general, the invention features compositions and methods for activating a neural receptor in a subject. The compositions and methods include formulations for administering a metabolic hormone to the subject without substantially changing the concentration of the metabolic hormone in the blood of the subject. Such methods may be used to treat addiction or a mood disorder. The invention is based, in part, upon the surprising discovery that administration of a metabolic hormone, such as PYY, GLP-1, leptin, amylin, insulin, or calcitonin, can activate a neural receptor in the central nervous system to treat the addiction or mood disorder. Certain neural receptors that can be targeted with the compositions and methods described herein include, for example, a Y receptor (YR), a Y1 receptor (Y1R), a Y2 receptor (Y2R), a Y4 receptor (Y4R), a Y5 receptor (Y5R), a G-protein coupled receptor (GPRCR), a leptin receptor (LEPR), a GLP-1 receptor (GLP-1R), an insulin receptor (INSR), a glucose-dependent insulinotropic polypeptide receptor (GIPR), a ghrelin receptor (GHS-R), a cholecystokinin A receptor (CCKAR), a cholecystokinin B receptor (CCKBR), or a calcitonin receptor (CALCR). By administering the metabolic hormone via one of the routes described herein, the composition can provide treatment to the subject without substantially changing the concentration of the agent in the blood of the subject. Furthermore, these routes of administration avoid systemic administration, which are known to produce unwanted side effects, such as nausea, injection site pain, or malaise. The compositions and methods are described in more detail below.

### Indications

The methods described herein include the administration of a composition containing a metabolic hormone as described herein to activate a neural receptor. Activation of the neural receptor provides treatment for an addiction or a mood disorder in the subject in need thereof. In general, by administering the metabolic hormone via a non-systemic pathway, the hormone can target specific pleasure centers in the brain, activate critical brain regions, and avoid neural or non-neural targets that can cause clinical risk (e.g., toxicity) or side effects, such as nausea. For example, systemic administration of a metabolic hormone activates neural receptors in the hypothalamus, nucleus tractus solitarius (NTS), and area postrema, whereas non-systemic routes of administration as described herein activates neural receptors in the hypothalamus and NTS, but substantially avoids the area postrema. By targeting neural receptors, e.g., in the mouth (e.g., on the tongue), nose, rectum, or GI tract, the neural receptors and connections can sufficiently activate the pleasure centers in the CNS. Accordingly, activation of these pleasure centers provides treatment for a disorder associated with dysregulated pleasure centers in the brain.

In one embodiment, the subject has a mood disorder (e.g., an affective disorder and/or psychiatric disorder). In some embodiments, the mood disorder is depression, anxiety, dysthymia, bipolar disorder, a medication-induced mood disorder, or a substance-induced mood disorder. In some embodiments, the depression includes major depression disorder (MDD), major depressive disorder with seasonal patterns (SAD), a type of depression due to hormonal changes (e.g., perinatal depression, postpartum depression, premenstrual dysphoric disorder). In some embodiments, anxiety includes panic attacks and/or generalized anxiety disorder. In some embodiments, the bipolar disorder includes bipolar disorder I, bipolar disorder II, and/or cyclothymia. In some embodiments, the psychiatric disorder includes mania, schizoaffective disorder, and schizophrenia. In some embodiments, the mood disorder includes developmental disorders (e.g., autism spectrum, attention deficit hyperactivity disorder, or attention deficit disorder), dementias (e.g., Alzheimer's disease or reversible dementias), personality disorders with fixed behavior patterns (e.g., depressive, schizoid, narcissistic, borderline disorder, or antisocial personality disorder), and problematic behaviors (e.g., shop lifting, lying, risk taking, impulse control difficulties, or adjustment disorders).

In some embodiments, the addiction or mood disorder does not include of post-traumatic stress disorder (PTSD), traumatic brain injury (TBI) Alzheimer's disease, memory loss, cognitive defects, stress, stroke, or a neurodegenerative disorder. In some embodiments, the method does not include administration of insulin. In some embodiments, the method does not include intranasal administration of insulin. In some embodiments, the method does not include intranasal administration of insulin for the treatment of Alzheimer's disease.

The subject may have an addiction, e.g., to a chemical substance. In some embodiments, the addiction includes a craving or a dependency for alcohol, cocaine, opioids, nicotine, heroin, marijuana, 3,4-methylenedioxy-methamphetamine, caffeine, mescaline, methamphetamine, amphetamine derivatives, dextromethorphan, loperamide, phenylcyclohexyl piperidine, stimulants, steroids, cannabinoids, cathinones, lysergic acid diethylamide, inhalants, sedatives, hypnotics, tobacco, anxiolytics, hallucinogens, food, or a combination thereof. In some embodiments, the method reduces the frequency or intensity of the craving (e.g., by at least 10%, 20%, 30%, 40%, 50%, 60%, 70%, 80%, 90%, 95%, or 100%). In some embodiments, the method eliminates the craving.

The subject may have a compulsive behavior (e.g., obsessive compulsive disorder) or addiction, such as an addiction to gambling, sex, repetitive behavior, or a destructive habit. In some embodiments, the method reduces the frequency of the behavior (e.g., by at least 10%, 20%, 30%, 40%, 50%, 60%, 70%, 80%, 90%, 95%, or 100%). In some embodiments, the method eliminates the behavior.

The subject may have a binge eating disorder.

The subject may be addicted to food. The subject may be addicted to eating (e.g., binge eating).

The compositions described herein may be used to activate hedonic (e.g., pleasure and/or reward) centers of the brain. Metabolic hormones (e.g., PYY, PYY(3-36), GLP-1, leptin, amylin, calcitonin, an analog, variant, or biologically active fragment thereof) may directly affect hedonic centers. Neural receptors targeted by the compositions described herein include Y receptors (e.g., Y1R, Y2R, Y4R, Y5R), a GPCR, LEPR, GLP-1R, INSR, GIPR, GHS-R, CCKAR, CCKBR, or CALCR. In some embodiments activation of neural receptors using the compositions described herein may occur via the gut-brain axis (e.g., vagal, spinal afferent neurons, cytokines, gut hormones, gut microbiota-derived signaling molecules).

### Metabolic Hormone Compositions

The compositions described herein include a metabolic hormone or a biologically active fragment, variant, or analog thereof. The metabolic hormone targets (e.g., binds, associates, or interacts with) a YR, a Y1R, a Y2R, a Y4R, a Y5R, a GPCR, a LEPR, a GLP-1R, an INSR, a GIPR, a GHS-R, a CCKAR, a CCKBR, or a CALCR in the subject. The receptor may be, for example, in an oral cavity (e.g., tongue), nasal cavity, GI tract, or rectum of a subject. In some embodiments, the metabolic hormone is PYY, PYY(3-36), leptin, amylin, insulin, calcitonin, or GLP-1, or a variant, analog, or biologically active fragment thereof.

In some embodiments, the dose of the metabolic hormone or a biologically active fragment, variant, or analog thereof is from 0.1 ng to 20 mg. For example, the dose of the agent may be from 0.1 ng to 1 ng, e.g., 0.2 ng, 0.3 ng, 0.4 ng, 0.5 ng, 0.6 ng, 0.7 ng, 0.8 ng, 0.9 ng, or 1 ng, e.g., 1 ng to 10 ng, e.g., 1 ng, 2 ng, 3 ng, 4 ng, 5 ng, 6 ng, 7 ng, 8 ng, 9 ng, or 10 ng, e.g., from 10 ng to 100 ng, e.g., 20 ng, 30 ng, 40 ng, 50 ng, 60 ng, 70 ng, 80 ng, 90 ng, or 100 ng, e.g., from 100 ng to 1 μg, e.g., 200 ng, 300 ng, 400 ng, 500 ng, 600 ng, 700 ng, 800 ng, 900 ng, or 1 μg, e.g., from 1 μg to 10 μg, e.g., 2 μg, 3, μg, 4 μg, 5 μg, 6 μg, 7 μg, 8 μg, 9 μg, or 10 μg, e.g., from 10 μg to 100 μg, e.g., 10 μg, 20 μg, 30 μg, 40 μg, 50 μg, 60 μg, 70 μg, 80 μg, 90 μg, or 100 μg, e.g., from 100 μg to 1 mg, e.g., 100 μg, 200 μg, 300 μg, 400 μg, 500 μg, 600 μg, 700 μg, 800 μg, 900 μg, or 1 mg, e.g., from 1 mg to 20 mg, e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg.

In some embodiments, the metabolic hormone is PYY or an analog, variant, or biologically active fragment thereof. In some embodiments, the PYY or variant, analog or biologically active fragment thereof has at least 70% (e.g., 70%, 75%, 80%, 85%, 90%, 95%, 97%, 99%, or 100%) sequence identity to SEQ ID NO: 1. In some embodiments, PYY or variant, analog, or biologically active fragment thereof has the amino acid sequence set forth in SEQ ID NO: 1. In some embodiments, the compositions described herein include PYY or variant, analog, or biologically active fragment thereof in a dose of from about 100 ng to about 10 mg. In some embodiments, the compositions described herein include PYY or variant, analog, or biologically active fragment thereof in a dose of from about 1 μg to about 1 mg. In some embodiments, the compositions described herein include PYY or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the PYY fragment is PYY(3-36). In some embodiments, the PYY(3-36) or variant, analog, or biologically active fragment thereof has at least 70% (e.g., at least 70%, 75%, 80%, 85%, 90%, 95%, 97%, 99%, or 100%) sequence identity to SEQ ID NO: 2. In some embodiments, PYY(3-36) has the amino acid sequence set forth in SEQ ID NO: 2. In some embodiments, the compositions described herein include PYY(3-36) or variant, analog, or biologically active fragment thereof in a dose of from about 100 ng to about 10 mg. In some embodiments, the compositions described herein include PYY(3-36) or variant, analog, or biologically active fragment thereof in a dose of from about 1 μg to about 1 mg. In some embodiments, the compositions described herein include PYY(3-36) or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the PYY variant is [Pro34]PYY. In some embodiments, the [Pro34]PYY or variant, analog, or biologically active fragment thereof has at least 70% (e.g., at least 70%, 75%, 80%, 85%, 90%, 95%, 97%, 99%, or 100%) sequence identity to SEQ ID NO: 11. In some embodiments, [Pro34]PYY has the amino acid sequence set forth in SEQ ID NO: 11. In some embodiments, the PYY analog is NNC065-1273, which contains the PYY(3-36) polypeptide with a beta-homo-arginine at position 35. In some embodiments, the PYY analog is NNC0165-1875. In some embodiments, the PYY analog is NNC0165-1562. In some embodiments, the PYY analog or variant is described, e.g., in Lear et al. J. of Med. Chem. 63:9660-9671, 2020, which is hereby incorporated by reference in its entirety. In some embodiments, the PYY analog is PYY-Ab (PYY conjugated to an antibody or an Fc region of an antibody) or PYY conjugated to one or more PEG moieties, e.g., as described in Rangwala et al. Cell Metab. 29:837-843, 2019, which is hereby incorporated by reference in its entirety. In some embodiments, the PYY analog or variant is described, e.g., in U.S. Pat. No. 8,217,001, the disclosure of which is hereby incorporated by reference in its entirety. In some embodiments, the compositions described herein include [Pro34]PYY or variant, analog, or biologically active fragment thereof in a dose of from about 100 ng to about 10 mg. In some embodiments, the compositions described herein include [Pro34]PYY or variant, analog, or biologically active fragment thereof in a dose of from about 1 μg to about 1 mg. In some embodiments, the compositions described herein include [Pro34]PYY or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the PYY(3-36) variant, analog, or biologically active fragment thereof is PYY(26-36), PYY(25-36), PYY(24-36), PYY(23-36), PYY(22-36), PYY(21-36), PYY(20-36), PYY(19-36), PYY(18-36), PYY(17-36), PYY(16-36), PYY(15-36), PYY(14-36), PYY(13-36), PYY(12-36), PYY(11-36), PYY(10-36), PYY(9-36), PYY(8-36), PYY(7-36), PYY(6-36), PYY(5-36), or PYY(4-36), as in Balasubramaniam et al., Pept Res 1:32-35, 1998; Liu et al., J. Gastrointest Surg. 5:147-152, 2001, herein incorporated by reference in their entirety. In some embodiments, the PYY(3-36) variant, analog, or fragment thereof may be a fragment with a single point mutation e.g., single point mutation of PYY(25-36) such as [Lys²⁵]PPY(25-36), [Thr²⁷]PPY(25-36), [Phe²¹]PPY(25-36), [Ile²⁸]PYY(25-36), [Val²⁸]PYY(25-36), [Gln²⁹]PYY(25-36), [Ile³⁰]PYY(25-36), [Val³⁰]PYY(25-36), [Ile³¹]PYY(25-36), [Leu³¹]PYY(25-36), [Ser³²]PYY(25-36), [Lys³³]PYY(25-36), [Asn³⁴]PYY(25-36), [Lys³⁵]PYY(25-36), [Thr³⁶]PYY(25-36), or [Phe³⁶]PYY(25-36) or a single point mutation of PYY(24-36) such as [Ile²⁴]PYY(24-36) or [Val24]PYY(24-36). In some embodiments, the PYY(3-36) variant, analog, or biologically active fragment thereof may be a fragment with a double point mutation e.g., double point mutation of PYY(25-36) such as [Lys25, Thr27]PPY(25-36), [Lys25, Phe27]PPY(25-36), [Lys25, Ile28]PPY(25-36), [Lys25, Val28]PPY(25-36), [Lys25, Gln29]PPY(25-36), [Lys²⁵, Ile³⁰]PPY(25-36), [Lys²⁵, Val³⁰]PPY(25-36), [Lys²⁵,Ile³¹]PPY(25-36), [Lys²⁵, Leu³¹]PPY(25-36), [Lys²⁵, Ser³²]PPY(25-36), [Lys²⁵, Lys³³]PPY(25-36), [Lys²⁵, Asn³⁴]PPY(25-36), [Lys²⁵, Lys³⁵]PPY(25-36), [Lys²⁵, Thr³⁶]PPY(25-36), [Lys²⁵, Phe³⁶]PPY(25-36), [Thr²⁷, Ile²⁸]PPY(25-36), [Thr²⁷, Val²⁸]PPY(25-36), [Thr²⁷, Gln²⁹]PPY(25-36), [Thr²⁷, Ile³⁰]PPY(25-36), [Thr²⁷, Val³⁰]PPY(25-36), [Thr²⁷, Ile³¹]PPY(25-36), [Thr²⁷, Leu³¹]PPY(25-36), [Thr²⁷, Ser³²]PPY(25-36), [Thr²⁷, Lys³³]PPY(25-36), [Thr²⁷, Asn³⁴]PPY(25-36), [Thr²⁷, Lys³⁵]PPY(25-36), [Thr²⁷, Thr³⁶]PPY(25-36), [Thr²⁷, Phe³⁶]PPY(25-36), [Phe²⁷, Ile²⁸]PPY(25-36), [Phe²⁷, Val²⁸]PPY(25-36), [Phe²⁷, Gln²⁹]PPY(25-36), [Phe²⁷, Ile³⁰]PPY(25-36), [Phe²⁷, Val³⁰]PPY(25-36), [Phe²⁷, Ile³¹]PPY(25-36), [Phe²⁷, Leu³¹]PPY(25-36), [Phe²⁷, Ser³²]PPY(25-36), [Phe²⁷, Lys³³]PPY(25-36), [Phe²⁷, Asn³⁴]PPY(25-36), [Phe²⁷, Lys³⁵]PPY(25-36), [Phe²⁷, Thr³⁶]PPY(25-36), [Phe²⁷, Phe³⁶]PPY(25-36), [Gln²⁹, Ile³⁰]PYY(25-36), [Gln²⁹, Val30]PYY(25-36), [Gln²⁹, Ile³¹]PYY(25-36), [Gln²⁹, Leu³¹]PYY(25-36), [Gln²⁹, Ser³²]PYY(25-36), [Gln²⁹, Leu³³]PYY(25-36), [Gln²⁹, Asn³⁴]PYY(25-36), [Gln²⁹, Leu³³]PYY(25-36), [Gln²⁹, Thr³⁶]PYY(25-36), [Gln²⁹, Phe³⁰]PYY(25-36), [Ile³⁰, Ile³¹]PYY(25-36), [Ile³⁰, Leu³¹]PYY(25-36), [Ile³⁰, Ser³²]PYY(25-36), [Ile³⁰, Lys³³]PYY(25-36), [Ile³⁰, Asn³⁴]PYY(25-36), [Ile³⁰, Lys³³]PYY(25-36), [Ile³⁰, Thr³⁰]PYY(25-36), [Ile³⁰, Phe³⁰]PYY(25-36), [Val³⁰, Ile³¹]PYY(25-36), [Val³⁰, Leu³¹]PYY(25-36), [Val³⁰, Ser³²]PYY(25-36), [Val³⁰, Lys³³]PYY(25-36), [Val³⁰, Asn³⁴]PYY(25-36), [Val³⁰, Lys³⁵]PYY(25-36), [Val³⁰, Thr³⁰]PYY(25-36), [Val³⁰, Phe³⁰]PYY(25-36), [Ile³¹, Ser³²]PYY(25-36), [Ile³¹, Lys³³]PYY(25-36), [Ile³¹, Asn³⁴]PYY(25-36), [Ile³¹, Lys³³]PYY(25-36), [Ile³¹, Thr³⁰]PYY(25-36), [Leu³¹, Phe³⁰]PYY(25-36), [Leu³¹, Ser³²]PYY(25-36), [Val³¹, Lys³³]PYY(25-36), [Leu³¹, Asn³⁴]PYY(25-36), [Leu³¹, Lys³³]PYY(25-36), [Leu³¹, Thr³⁰]PYY(25-36), [Leu³¹, Phe³⁰]PYY(25-36), [Ser³², Lys³³]PYY(25-36), [Ser³², Asn³⁴]PYY(25-36), [Ser³², Lys³⁵]PYY(25-36), [Ser³², Thr³⁶]PYY(25-36), [Ser³², Phe³⁶]PYY(25-36), [Lys³³, Asn³⁴]PYY(25-36), [Lys³³, Lys³⁵]PYY(25-36), [Lys³³, Thr³⁶]PYY(25-36), [Lys³³, Phe³⁶]PYY(25-36), [Asn³⁴, Lys³⁵]PYY(25-36), [Asn³⁴, Thr³⁶]PYY(25-36), [Asn³⁴, Phe³⁶]PYY(25-36), [Lys³⁵, Thr³⁶]PYY(25-36), or [Lys³⁵, Phe³⁶]PYY(25-36).

In some embodiments, the metabolic hormone is leptin or an analog, variant, or biologically active fragment thereof. In some embodiments, the leptin or variant, analog, or biologically active fragment thereof has at least 70% (e.g., at least 70%, 75%, 80%, 85%, 90%, 95%, 97%, 99%, or 100%) sequence identity to SEQ ID NO: 6. In some embodiments, leptin has the amino acid sequence set forth in SEQ ID NO: 6. In some embodiments, the analog of leptin is the recombinant analog metreleptin (MYALEPT®), which contains a polypeptide having the sequence set forth in SEQ ID NO: 9 and contains a disulfide bridge connecting amino acid residues 97 and 147. In some embodiments, the analog of leptin is a murine leptin analog, e.g., as described in Peters et al. Endocrinol. 148: 2878-2885, 2007, which is hereby incorporated by reference in its entirety. In some embodiments, the compositions described herein include leptin or variant, analog, or biologically active fragment thereof in a dose of from about 100 ng to about 10 mg. In some embodiments, the compositions described herein include leptin or variant, analog, or biologically active fragment thereof in a dose of from about 1 μg to about 1 mg. In some embodiments, the compositions described herein include leptin or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is amylin or an analog (e.g., pramlintide (SYMLIN®)), variant, or biologically active fragment thereof. In some embodiments, the amylin or variant, analog, or biologically active fragment thereof has at least 70% (e.g., at least 70%, 75%, 80%, 85%, 90%, 95%, 97%, 99%, or 100%) sequence identity to SEQ ID NO: 7. In some embodiments, amylin has the amino acid sequence set forth in SEQ ID NO: 7. In some embodiments, the analog of amylin is pramlintide (SYMLIN®), which contains a polypeptide having to the sequence set forth in SEQ ID NO: 8. In some embodiments, the compositions described herein include amylin or variant, analog, or biologically active fragment thereof in a dose of from about 100 ng to about 10 mg. In some embodiments, the compositions described herein include amylin or variant, analog, or biologically active fragment thereof in a dose of from about 1 μg to about 1 mg. In some embodiments, the compositions described herein include amylin or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is GLP-1 or an analog, variant, or biologically active fragment thereof. In some embodiments, the GLP-1 or variant, analog, or biologically active fragment thereof has at least 70% (e.g., at least 70%, 75%, 80%, 85%, 90%, 95%, 97%, 99%, or 100%) sequence identity to SEQ ID NO: 3. In some embodiments, GLP-1 has the amino acid sequence set forth in SEQ ID NO: 3. In some embodiments, the GLP-1 fragment is GLP-1(7-36) or variant, analog, or biologically active fragment thereof. In some embodiments, GLP-1(7-36) or variant, analog, or biologically active fragment thereof has at least 70% (e.g., at least 70%, 75%, 80%, 85%, 90%, 95%, 97%, 99%, or 100%) sequence identity to SEQ ID NO: 4. In some embodiments, GLP-1(7-36) has the amino acid sequence set forth in SEQ ID NO: 4. In some embodiments, the GLP-1 fragment is GLP-1(7-37) or variant, analog, or biologically active fragment thereof. In some embodiments, GLP-1(7-37) or variant, analog, or biologically active fragment thereof has at least 70% (e.g., at least 70%, 75%, 80%, 85%, 90%, 95%, 97%, or 100%) sequence identity to SEQ ID NO: 5. In some embodiments, GLP-1(7-37) has the amino acid sequence set forth in SEQ ID NO: 5. In some embodiments, the compositions described herein include GLP-1 or variant, analog, or biologically active fragment thereof in a dose of from about 100 ng to about 10 mg. In some embodiments, the compositions described herein include GLP-1 or variant, analog, or biologically active fragment thereof in a dose of from about 1 μg to about 1 mg. In some embodiments, the compositions described herein include GLP-1 or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is calcitonin or an analog, variant, or biologically active fragment thereof. In some embodiments, calcitonin or variant, analog, or biologically active fragment thereof has at least 70% (e.g., 70%, 75%, 80%, 85%, 90%, 95%, 97%, 99%, or 100%) sequence identity to SEQ ID NO: 10. In some embodiments, calcitonin has the amino acid sequence set forth in SEQ ID NO: 10. In some embodiments, the compositions described herein include calcitonin or variant, analog, or biologically active fragment thereof in a dose of from about 100 ng to about 10 mg. In some embodiments, the compositions described herein include calcitonin or variant, analog, or biologically active fragment thereof in a dose of from about 1 μg to about 1 mg. In some embodiments, the compositions described herein include calcitonin or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is insulin, or an analog (e.g., insulin aspart (NOVOLOG®), insulin glargine (LANTUS®), insulin lispro (LYUMJEV™), insulin glulisine (APIDRA®), or insulin detemir (LEVEMIR®), insulin degludec (TRESIBA®), NPH insulin (HUMULIN® N or NOVOLIN® N), a variant, or biologically active fragment thereof. In some embodiments, insulin or a variant, analog, or biologically active fragment thereof has at least 70% (e.g., at least 70%, 75%, 80%, 85%, 90%, 95%, 97%, 99%, or 100%) sequence identity to SEQ ID NO: 12. In some embodiments, insulin has the amino acid sequence set forth in SEQ ID NO: 12. In some embodiments, the analog of insulin is insulin aspart (NOVOLOG®), having an A chain with the sequence set forth in SEQ ID NO: 13 and a B chain with the sequence set forth in SEQ ID NO: 14. In some embodiments, the analog of insulin is insulin glargine (LANTUS®), having an A chain with sequence set forth in SEQ ID NO: 15 and a B chain with to the sequence set forth in SEQ ID NO: 16. In some embodiments, the analog of insulin is insulin lispro (LYUMJEV™), having an A chain with the sequence set forth in SEQ ID NO: 17 and a B chain with to the sequence set forth in SEQ ID NO: 18. In some embodiments, the analog of insulin is insulin glulisine (APIDRA®), having an A chain with the sequence set forth in SEQ ID NO: 19 and a B chain with to the sequence set forth in SEQ ID NO: 20. In some embodiments, the analog of insulin is insulin detemir (LEVEMIR®), having an A chain with to the sequence set forth in SEQ ID NO: 21 and a B chain with to the sequence set forth in SEQ ID NO: 22. In some embodiments, the methods herein described include the topical-lingual (e.g., topically to the lingual epithelium) administration of insulin or a variant, analog, or biologically active fragment thereof in a dose of from about 2.5 μg to about 2.5 mg. In some embodiments, the methods herein described include the topical-lingual administration of insulin or a variant, analog, or biologically active fragment thereof in a dose of from about 10 μg to about 1 mg. In some embodiments, the methods herein described include the topical-lingual administration of insulin or a variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

### Pharmaceutical Compositions and Routes of Administration

The metabolic hormones or variants, analogs, or biologically active fragments thereof described herein can be formulated as pharmaceutical compositions for administration to human subjects in a biologically compatible form suitable for administration in vivo.

The compositions described herein may be administered to a subject (e.g., a human) in a variety of forms depending on the selected route of administration, as will be understood by those skilled in the art. The compositions described herein may be administered, for example, by any route that allows the composition (e.g., the metabolic hormone) to reach the target receptor without substantially changing the concentration of the metabolic hormone in the blood of the subject. The composition may be administered by, for example, topical-lingual (e.g., topically to the lingual epithelium), intranasal, intrarectal, or topical GI routes.

In some embodiments, the compositions described herein are formulated for delivery to the oral cavity, e.g., intraoral, oromucosal, transmucosal, topical lingual, gargles, mouthwashes, gingival solutions, oromucosal solutions and oromucosal suspensions, semi-solid oromucosal preparations (including for example gingival gel, gingival paste, oromucosal gel, oromucosal paste), oromucosal drops, oromucosal sprays and sublingual sprays (including oropharyngeal sprays), dry powder sprays, lozenges and pastilles, compressed lozenges, sublingual tablets and buccal tablets, oromucosal capsules, mucoadhesive preparations). See, e.g., Oromucosal Preparations, (Ph Eur monograph 1807). In some embodiments, the metabolic hormone in the pharmaceutical composition is adapted for binding to the Y2 receptors expressed in the oral cavity (e.g., on the tongue).

In some embodiments, the metabolic hormone is formulated as a lozenge, a film, a spray, or an orally dissolvable tablet (ODT). In some embodiments, the metabolic hormone is formulated as an ODT using a formulation that rapidly dissolves on the tongue of a subject. For example, the formulation may have partially hydrolyzed gelatin at a concentration of from about 1% to 6% w/v (e.g., about 1%, about 2%, about 3%, about 4%, about 5%, or about 6%), mannitol, hydrolyzed dextran, alginate, polyvinyl alcohol, polyvinylpyrrolidone, acacia, aspartame, sodium methylparaben, sodium propylparaben, phenylalanine, water, or a combination thereof. In some embodiments, the ODT is formulated using the ZYDIS® formulation, as described in U.S. Pat. Nos. 4,305,502, 4,371,516, and 5,738,875, herein incorporated in their entirety by reference.

In some embodiments, the compositions described herein are formulated for intranasal delivery. The intranasal composition may be formulated, e.g., as a spray, a semisolid, a particular, or in a lipid-based carrier. The formulation may be, e.g., a solution, a suspension, a powder, or a gel. Suitable intranasal formulations are described, e.g., in Marx et al. Drug Discov Dev299-320, 2015, which is hereby incorporated by reference in its entirety. In some preferred embodiments, the compositions described herein are administered via inhalation, e.g., via nasal inhalation. An inhalable composition described herein may be provided as a liquid dosage form or dry powder dosage form. A dry powder composition may be, e.g., administered by inhalation as is or after reconstitution in a vehicle, e.g., saline (e.g., isotonic saline), phosphate-buffered saline, or water. In some embodiments, the intranasal formulation does not include insulin.

In some embodiments, the compositions described herein are formulated for topical administration to the GI tract. The topical composition may be formulated, e.g., as a GI patch. Suitable GI patch formulations are described, e.g., in Tao, et al Drug discovery Today 10:909-915, 2005, which is hereby incorporated by reference it its entirety.

In some embodiments, the compositions described herein are formulated for intrarectal delivery. The intrarectal composition may be formulated, e.g., as a suppository, an enema, an ointment, or a rectal foam. Suitable intrarectal formulations are described, e.g., in Hua Front. Pharmacol. 10: 1196, 2019, which is hereby incorporated by reference in its entirety. Compositions for rectal administration may be in the form of suppositories containing a conventional suppository base, such as cocoa butter.

Solutions of a composition described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018. The composition described herein may be administered to an animal, e.g., a human, alone or in combination with pharmaceutically acceptable carriers, as noted herein, the proportion of which is determined by the solubility and chemical nature of the composition, chosen route of administration, and standard pharmaceutical practice.

In some embodiments, the compositions include excipients that increase the time the metabolic hormone, e.g., PYY(3-36), is in contact with the mucosa (e.g., oral mucosa, nasal mucosa, GI mucosa, or rectal mucosa). The excipients may provide viscosity enhancement, encapsulation, and controlled release. Without being bound by theory, it is believed that increasing the contact time of the pharmaceutical formulation with the mucosa, leads to increased binding of the metabolic hormone to its receptor. Suitable excipients for viscosity enhancement include rheology modifiers which also may be mucoadhesive such as methylcellulose, hydroxyethylcellulose, hydroxypropylcellulose, alginic acid, polyvinylpyrrolidone, and sodium carboxymethylcellulose. Suitable excipients for modified release of the metabolic hormone in the mucosa include mucoadhesive permeation enhancers such as 23-lauryl ether, aprotinin, azone, benzalkonium chloride, cetylpyridinium chloride, cetyltrimethylammonium bromide, cyclodextrins, dextran sulfate, and lauric acid. Other suitable mucoadhesive polymers used for the compositions described herein include agarose, chitosan, gelatin, hyaluronic acid, gums (e.g. guar, hakea, xanthan, gellan, carrageenan, pectin, and sodium alginate), cellulose derivatives (e.g., CMC, thiolated CMC, sodium CMC, HEC, HPMC, MC, methylhydroxylethylcellulose), poly (acrylic acid)-based polymers (e.g., CP, PC, PAA, polyacrylates, poly(methylvinylether-co-methacrylic acid), poly(2-hydroxyethyl methacryalate), poly(alkylcyanoacryalate), poly(isohexylcyanocrylate), poly(isobutylcyanoacrylate), copolymer of acrylic acid and PEG, poly(N-2-hydroxypropyl methacrylamide), PHPMAm, polyoxyethylene, PVA, PVP, and other thiolated polymers; scleroglucan, PVA, steroidal detergents, non-ionic surfactants, laureth-9, sodium fusidate, included sodium lauryl, sodium laurate (e.g., pH 8.9), palmitoyl carnitine, lauric acid/propylene glycol vehicle, Brij 78, sodium deoxycholate, sodium lauryl sulfate, lecithin and PVP. See, e.g., International Journal of Pharmaceutics, Volume 53, Issue 3, 1 Aug. 1989, Pages 227-235.

In some embodiments, the pharmaceutical compositions include excipients that increase the residence time of a metabolic hormone in the saliva (e.g., the amount of time the metabolic hormone remains in the saliva without significant degradation of the peptide). Without being bound by theory, it is believed that increasing the residence time of the metabolic hormone in the saliva increases the opportunity for the metabolic hormone to bind its receptor on the tongue. The residence time in the saliva can optionally be adjusted to avoid increasing systemic exposure to the metabolic hormone through, for example, swallowing.

A composition containing a metabolic hormone as described herein can include one or more pharmaceutically acceptable excipients, such as propylene glycol, potassium sorbate, I-arginine, edetate disodium, monosodium phosphate, and polysorbate 20. In some embodiments, propylene glycol is present in a concentration of about 100 mg/ml, I-arginine is present in a concentration of about 25 mg/ml, potassium sorbate is present in a concentration of about 2 mg/ml, edetate disodium is present in a concentration of about 1.2 mg/ml, sodium phosphate monobasic dihydrate is present in a concentration of about 7.8 mg/ml, and polysorbate is present in a concentration of about 5 mg/ml. Furthermore, the compositions described herein can include co-solvent stabilizers like propylene glycol or other suitable co-solvent stabilizers (e.g., lower molecular weight polyethylene glycols (PEG) such as PEG 200 and 400, glycerin, and ethanol. In some embodiments, compositions described herein include amino acid stabilizers like L-arginine or other suitable amino acid stabilizers (e.g., alanine, aspartic acid, glycine, lysine, proline, or methionine). In some embodiments, compositions described herein can include preservatives like potassium sorbate, or other suitable preservatives (e.g., ascorbic acid, benzyl alcohol, benzoic acid, citric acid, chlorobutanol, m-cresol, glutathione, methionine, methylparaben, propylparaben, sodium sulfite, parahydroxybenzoate esters (methylhydroxybenzoate and propylhydroxybenzoate), boric acid and borate salts, sorbic acid and other sorbate salts besides potassium, and phenolics). compositions described herein can include antioxidants such as edetate disodium or another suitable antioxidant (e.g., sodium formaldehyde sulphoxylate, butylated hydroxyanisole, and butylated hydroxytoluene). In some embodiments, the compositions described herein include buffers (e.g., acetate, carbonate, citrate, citrate-phosphate, glycine, HEPES, histidine, maleate, phosphate, succinate, tartrate, and triethanolamine (Tris)). In some embodiments, the compositions described herein can include surfactants, such as polysorbate 20 or other suitable surfactants (e.g., Poloxamer 188/407, polysorbate 40 or 80, or sodium lauryl sulfate).

In some embodiments, the excipients include flavorings to increase compliance with ingesting the composition. For example, the flavorings can be used to mask bitter or other undesirable flavor properties, or to make the composition compatible with the flavor of food that may be ingested before or after administration of the composition. Compatible flavorings include, for example, apple, banana, bubblegum, cherry, chocolate, grape, lemon, mango, orange, raspberry, strawberry, vanilla, watermelon, mint or a combination of the above flavors. In another aspect, these flavorings are dye-free, sugar-free, hypoallergenic, gluten-free, and casein-free.

In some embodiments, the pharmaceutical composition may be administered as in a unit dose form or as a dose per mass or weight of the patient from 0.01 ng/kg to 250 μg/kg (e.g., for a person of 75 kg body weight). For example, the dose of the metabolic hormone may be from 0.01 ng/kg to 0.1 ng/kg, e.g., 0.01 ng/kg, 0.02 ng/kg, 0.03 ng/kg, 0.04 ng/kg, 0.05 ng/kg, 0.06 ng/kg, 0.07 ng/kg, 0.08 ng/kg, 0.09 ng/kg, or 0.1 ng/kg, e.g., from 0.1 ng/kg to 1 ng/kg, e.g., 0.1 ng/kg, 0.2 ng/kg, 0.3 ng/kg, 0.4 ng/kg, 0.5 ng/kg, 0.6 ng/kg, 0.7 ng/kg, 0.8 ng/kg, 0.9 ng/kg, or 1 ng/kg, e.g., from 1 ng/kg to 10 ng/kg, e.g., 1 ng/kg, 2 ng/kg, 3 ng/kg, 4 ng/kg, 5 ng/kg, 6 ng/kg, 7 ng/kg, 8 ng/kg, 9 ng/kg, or 10 ng/kg, e.g., from 10 ng/kg to 100 ng/kg, e.g., 10 ng/kg, 20 ng/kg, 30 ng/kg, 40 ng/kg, 50 ng/kg, 60 μg/kg, 70 ng/kg, 80 ng/kg, 90 ng/kg, or 100 ng/kg, e.g., from 100 ng/kg to 1 μg/kg, e.g., 100 ng/kg, 200 ng/kg, 300 ng/kg, 400 ng/kg, 500 ng/kg, 600 ng/kg, 700 ng/kg, 800 ng/kg, 900 ng/kg, or 1 μg/kg, e.g., from 1 μg/kg to 10 μg/kg, e.g., 1 μg/kg, 2 μg/kg, 3 μg/kg, 4 μg/kg, 5 μg/kg, 6 μg/kg, 7 μg/kg, 8 μg/kg, 9 μg/kg, or 10 μg/kg, or e.g., from 10 μg/kg to 250 μg/kg, e.g., 10 μg/kg, 20 μg/kg, 30 μg/kg, 40 μg/kg, 50 μg/kg, 60 μg/kg, 70 μg/kg, 80 μg/kg, 90 ng/kg, 100 μg/kg, 110 μg/kg, 120 μg/kg, 130 μg/kg, 140 μg/kg, 150 μg/kg, 160 μg/kg, 170 μg/kg, 180 μg/kg, 190 μg/kg, 200 μg/kg, 210 μg/kg, 220 μg/kg, 230 μg/kg, 240 μg/kg, or 250 μg/kg.

In general, the dosage of a pharmaceutical composition or the active agent (e.g., metabolic hormone, e.g., PYY (e.g., PYY(3-36)), GLP-1, leptin, amylin, insulin, or calcitonin, or an analog, variant, or biologically active fragment thereof) in a pharmaceutical composition may be in the range of from about 10 ng to about 200 μg per kg body weight, e.g., from about 100 ng to about 10 μg per kg body weight, or e.g., from about 100 ng to about 2.5 μg per kg body weight, e.g., a dose of about 100 ng, 200 ng, 300 ng, 400 ng, 500 ng, 600 ng, 700 ng, 800 ng, 900 ng, 1 μg, 2 μg, or 2.5 μg per kg body weight (e.g., for a person of 75 kg body weight).

Furthermore, it is understood that the dosage of an analog, variant, or biologically active fragment of the active agent may be administered as a molar equivalent amount of the active agent. The skilled artisan would understand, for example, that a metabolic hormone analog containing a posttranslational modification or a half-life extending moiety may require an increased (e.g., by 5%, 10%, 15%, 20%, 25%, 30%, 40%, 50%, 60%, 70%, 80%, 90%, 100%, 200%, 300%, or more) dosage than the dosage of the corresponding metabolic hormone without the posttranslational modification or half-life extending moiety.

The pharmaceutical composition may also be administered as a dose per mass or weight of the patient per unit day (e.g., 0.01 ng/kg/day to 250 μg/kg/day). In some embodiments, the pharmaceutical composition is administered at a dose from 10 ng/kg/day to 200 μg/kg/day (e.g., from 50 ng/kg/day to 100 μg/kg/day, from 100 ng/kg/day to 50 μg/kg/day, from 500 ng/kg/day to 1 μg/kg/day). In some embodiments, the pharmaceutical composition is administered at a dose from 100 ng/kg/day to 10 μg/kg/day (e.g., 100 ng/kg/day, 110 ng/kg/day, 120 ng/kg/day, 130 ng/kg/day, 140 ng/kg/day, 150 ng/kg/day, 160 ng/kg/day, 170 ng/kg/day, 180 ng/kg/day, 190 ng/kg/day, 200 ng/kg/day, 210 ng/kg/day, 220 ng/kg/day, 230 ng/kg/day, 240 ng/kg/day, 250 ng/kg/day, 260 ng/kg/day, 270 ng/kg/day, 280 ng/kg/day, 290 ng/kg/day, 300 ng/kg/day, 310 ng/kg/day, 320 ng/kg/day, 330 ng/kg/day, 340 ng/kg/day, 350 ng/kg/day, 360 ng/kg/day, 370 ng/kg/day, 380 ng/kg/day, 390 ng/kg/day, 400 ng/kg/day, 410 ng/kg/day, 420 ng/kg/day, 430 ng/kg/day, 440 ng/kg/day, 450 ng/kg/day, 460 ng/kg/day, 470 ng/kg/day, 480 ng/kg/day, 490 ng/kg/day, 500 ng/kg/day, 510 ng/kg/day, 520 ng/kg/day, 530 ng/kg/day, 540 ng/kg/day, 550 ng/kg/day, 560 ng/kg/day, 570 ng/kg/day, 580 ng/kg/day, 590 ng/kg/day, 600 ng/kg/day, 610 ng/kg/day, 620 ng/kg/day, 630 ng/kg/day, 640 ng/kg/day, 650 ng/kg/day, 660 ng/kg/day, 670 ng/kg/day, 680 ng/kg/day, 690 ng/kg/day, 700 ng/kg/day, 710 ng/kg/day, 720 ng/kg/day, 730 ng/kg/day, 740 ng/kg/day, 750 ng/kg/day, 760 ng/kg/day, 770 ng/kg/day, 780 ng/kg/day, 790 ng/kg/day, 800 ng/kg/day, 810 ng/kg/day, 820 ng/kg/day, 830 ng/kg/day, 840 ng/kg/day, 850 ng/kg/day, 860 ng/kg/day, 870 ng/kg/day, 880 ng/kg/day, 890 ng/kg/day, 900 ng/kg/day, 910 ng/kg/day, 920 ng/kg/day, 930 ng/kg/day, 940 ng/kg/day, 950 ng/kg/day, 960 ng/kg/day, 970 ng/kg/day, 980 ng/kg/day, 990 ng/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day, or 10 μg/kg/day). In some embodiments, the pharmaceutical composition is administered at a dose from about 100 ng/kg/day to about 2.5 μg/kg/day (e.g., 100 ng/kg/day, 110 ng/kg/day, 120 ng/kg/day, 130 ng/kg/day, 140 ng/kg/day, 150 ng/kg/day, 160 ng/kg/day, 170 ng/kg/day, 180 ng/kg/day, 190 ng/kg/day, 200 ng/kg/day, 210 ng/kg/day, 220 ng/kg/day, 230 ng/kg/day, 240 ng/kg/day, 250 ng/kg/day, 260 ng/kg/day, 270 ng/kg/day, 280 ng/kg/day, 290 ng/kg/day, 300 ng/kg/day, 310 ng/kg/day, 320 ng/kg/day, 330 ng/kg/day, 340 ng/kg/day, 350 ng/kg/day, 360 ng/kg/day, 370 ng/kg/day, 380 ng/kg/day, 390 ng/kg/day, 400 ng/kg/day, 410 ng/kg/day, 420 ng/kg/day, 430 ng/kg/day, 440 ng/kg/day, 450 ng/kg/day, 460 ng/kg/day, 470 ng/kg/day, 480 ng/kg/day, 490 ng/kg/day, 500 ng/kg/day, 510 ng/kg/day, 520 ng/kg/day, 530 ng/kg/day, 540 ng/kg/day, 550 ng/kg/day, 560 ng/kg/day, 570 ng/kg/day, 580 ng/kg/day, 590 ng/kg/day, 600 ng/kg/day, 610 ng/kg/day, 620 ng/kg/day, 630 ng/kg/day, 640 ng/kg/day, 650 ng/kg/day, 660 ng/kg/day, 670 ng/kg/day, 680 ng/kg/day, 690 ng/kg/day, 700 ng/kg/day, 710 ng/kg/day, 720 ng/kg/day, 730 ng/kg/day, 740 ng/kg/day, 750 ng/kg/day, 760 ng/kg/day, 770 ng/kg/day, 780 ng/kg/day, 790 ng/kg/day, 800 ng/kg/day, 810 ng/kg/day, 820 ng/kg/day, 830 ng/kg/day, 840 ng/kg/day, 850 ng/kg/day, 860 ng/kg/day, 870 ng/kg/day, 880 ng/kg/day, 890 ng/kg/day, 900 ng/kg/day, 910 ng/kg/day, 920 ng/kg/day, 930 ng/kg/day, 940 ng/kg/day, 950 ng/kg/day, 960 ng/kg/day, 970 ng/kg/day, 980 ng/kg/day, 990 ng/kg/day, 1 μg/kg/day, 1.1 μg/kg/day, 1.2 μg/kg/day, 1.3 μg/kg/day, 1.4 μg/kg/day, 1.5 μg/kg/day, 1.6 μg/kg/day, 1.7 μg/kg/day, 1.8 μg/kg/day, 1.9 μg/kg/day, 2 μg/kg/day, 2.1 μg/kg/day, 2.2 μg/kg/day, 2.3 μg/kg/day, 2.4 μg/kg/day, or 2.5 μg/kg/day).

The dosage of the compositions (e.g., a composition including a metabolic hormone) described herein, can vary depending on many factors, such as the pharmacodynamic properties of the metabolic hormone, the mode of administration, the age, health, and weight of the recipient, the nature and extent of the symptoms, the frequency of the treatment, and the type of concurrent treatment, if any, and the clearance rate of the composition in the animal to be treated. The compositions described herein may be administered initially in a suitable dosage that may be adjusted as required, depending on the clinical response. In some embodiments, the dosage of a composition (e.g., a composition including a metabolic hormone) is a prophylactically or a therapeutically effective amount. Furthermore, it is understood that all dosages may be continuously given or divided into dosages given per a given time frame. The composition can be administered, for example, every hour, day, week, month, or year. In some embodiments, the composition may be administered continuously. For example, a rectal formulation or GI patch may be present on the subject for a sustained amount of time (e.g., for at least 1 hour, 2 hours, 3 hours, 4 hours, 5 hours, 6 hours, 12 hours, 24 hours, 2 days, 3 days, 4 days, 5 days, 6 days, 7 days, 2 weeks, 3 weeks, 4 weeks, 2 months, 3 months, or longer).

The pharmaceutical compositions described herein (e.g., containing a metabolic hormone) may be provided in a kit that includes the pharmaceutical composition (e.g., in a container) and instructions for use thereof. The kit may contain one or more containers, in which each container contains a different composition of the invention. The instructions enclosed with the kit may be used to instruct a user to perform a method as described herein.

The methods described herein include administration of a metabolic hormone (e.g., PYY, PYY(3-36), leptin, amylin, insulin, GLP-1, or an analog, variant, or biologically active fragment thereof locally to the mouth (e.g., tongue, salivary glands, lingual and/or sublingual epithelium, or mucosa), rectum, nasal cavity, or GI tract of a subject, wherein the local administration does not produce substantial change to the level of the metabolic hormone in the blood and/or plasma of the subject. In general, the metabolic hormone level in the blood and/or plasma of the subject does not increase more than up to 10% (e.g., 9%, 8%, 7%, 6%, 5%, 4%, 3%, 2%, 1%, or less) of the pre-administration level of metabolic hormone. For example, PYY administered to a subject topical-lingually (e.g., topically to the lingual epithelium), at any dosage herein described, would reach at most a peak level of one tenth that of the endogenous level of PYY and decrease substantially thereafter. In some embodiments, the blood and/or plasma level of PYY(3-36) does not substantially surpass pre-prandial levels of from about 15 pmol/l to about 25 pmol/l as reported in Batterham et al, Cell Metabolism, 4:223-233, 2006, herein incorporated by reference, in its entirety, after topical-lingual administration of PYY(3-36). In some embodiments, the blood and/or plasma level of leptin does not substantially surpass pre-prandial levels of from about 5 ng/ml to about 35 ng/ml as reported in Considine et al, N. Engl. J. Med. 334:292-295, 1996, herein incorporated by reference in its entirety, after topical-lingual (e.g., topically to the lingual epithelium) administration of leptin. In some embodiments, the blood and/or plasma level of amylin does not substantially surpass pre-prandial levels of about 20 pmol/l as reported in Cooper et al, Hypertension, 26:460-464, 1995, herein incorporated by reference in its entirety, after topical-lingual (e.g., topically to the lingual epithelium) administration of amylin.

### Sequences

PYY
SEQ ID NO: 1
MVFVRRPWPALTTVLLALLVCLGALVDAYPIKPEAPREDASPEELNRYYA
SLRHYLNLVTRQRYGKRDGPDTLLSKTFFPDGEDRPVRSRSEGPDLW
PYY(3-36)
SEQ ID NO: 2
IKPEAPGEDASPEELNRYYASLRHYLNLVTRQRY
GLP-1
SEQ ID NO: 3
MKSIYFVAGLFVMLVQGSWQRSLQDTEEKSRSFSASQADPLSDPDQMNED
KRHSQGTFTSDYSKYLDSRRAQDFVQWLMNTKRNRNNIAKRHDEFERHAE
GTFTSDVSSYLEGQAAKEFIAWLVKGRGRRDFPEEVAIVEELGRRHADGS
FSDEMNTILDNLAARDFINWLIQTKITDRK
GLP-1(7-36)
SEQ ID NO: 4
HAEGTFTSDVSSYLEGQAAKEFIAWLVKGR
GLP-1(7-37)
SEQ ID NO: 5
HAEGTFTSDVSSYLEGQAAKEFIAWLVKGRG
Leptin
SEQ ID NO: 6
MHWGTLCGFLWLWPYLFYVQAVPIQKVQDDTKTLIKTIVTRINDISHTQS
VSSKQKVTGLDFIPGLHPILTLSKMDQTLAVYQQILTSMPSRNVIQISND
LENLRDLLHVLAFSKSCHLPWASGLETLDSLGGVLEASGYSTEVVALSRL
QGSLQDMLWQLDLSPGC
Amylin
SEQ ID NO: 7
MGILKLQVFLIVLSVALNHLKATPIESHQVEKRKCNTATCATQRLANFLV
HSSNNFGAILSSTNVGSNTYGKRNAVEVLKREPLNYLPL
Pramlintide
SEQ ID NO: 8
KCNTATCATQRLANFLVHSSNNFGPILPPTNVGSNTY
Metreleptin
SEQ ID NO: 9
MVPIQKVQDDTKTLIKTIVTRINDISHTQSVSSKQKVTGLDFIPGLHPIL
TLSKMDQTLAVYQQILTSMPSRNVIQISNDLENLRDLLHVLAFSKSCHLP
WASGLETLDSLGGVLEASGYSTEVVALSRLQGSLQDMLWQLDLSPGC
(Disulfide bridge: 97-147)
Calcitonin
SEQ ID NO: 10
CGNLSTCMLGTYTQDFNKFHTFPQTAIGVGAP
[Pro34]PYY
SEQ ID NO: 11
YPIKPEAPGEDASPEELNRYYASLRHYLNLVTRPRY
Insulin
SEQ ID NO: 12
MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN
Insulin Aspart
A chain
SEQ ID NO: 13
GIVEQCCTSICSLYQLENYCN
Insulin Aspart
B chain
SEQ ID NO: 14
FVNQHLCGSHLVEALYLVCGERGFFYTDKT
Insulin Glargine
A chain
SEQ ID NO: 15
GIVEQCCTSICSLYQLENYCG
Insulin Glargine
B chain
SEQ ID NO: 16
FVNQHLCGSHLVEALYLVCGERGFFYTPKTRR
Insulin Lispro
A chain
SEQ ID NO: 17
GIVEQCCTSICSLYQLENYCN
Insulin Lispro
B chain
SEQ ID NO: 18
FVNQHLCGSHLVEALYLVCGERGFFYTKPT
Insulin Glulisine
A chain
SEQ ID NO: 19
GIVEQCCTSICSLYQLENYCN
Insulin Glulisine
B chain
SEQ ID NO: 20
FVKQHLCGSHLVEALYLVCGERGFFYTPET
Insulin Detemir
A chain
SEQ ID NO: 21
GIVEQCCTSICSLYQLENYCN
Insulin Detemir
B chain
SEQ ID NO: 22
FVNQHLCGSHLVEALYLVCGERGFFYTPK

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A method of activating a neural receptor in a subject, the method comprising the step of administering to the subject a composition comprising an agent selected from Peptide YY (PYY), glucagon-like peptide 1 (GLP-1), leptin, amylin, insulin, and calcitonin, or an analog, variant, or biologically active fragment thereof, wherein the composition activates the neural receptor without substantially changing the concentration of the agent in the blood of the subject.

2. The method of claim 1, wherein activation of the neural receptor provides treatment for an addiction or a mood disorder in the subject in need thereof.

3. The method of claim 2, wherein the mood disorder comprises depression, anxiety, dysthymia, bipolar disorder, a medication-induced mood disorder, obsessive compulsive disorder, binge eating disorder, a substance-induced mood disorder, major depression disorder, major depressive disorder with seasonal patterns, a type of depression due to hormonal changes, panic attacks, generalized anxiety disorder, bipolar disorder I, bipolar disorder II, cyclothymia, mania, schizoaffective disorder, schizophrenia, autism spectrum, attention deficit hyperactivity disorder, attention deficit disorder, dementia, Alzheimer's disease, reversible dementia, depressive personality disorder, schizoid personality disorder, narcissistic personality disorder, borderline disorder, antisocial personality disorder, shop lifting, lying, risk taking, impulse control difficulty, or an adjustment disorder.

4. The method of claim 2 or 3, wherein the addiction comprises a craving or a dependency for alcohol, cocaine, opioids, nicotine, heroin, marijuana, 3,4-methylenedioxy-methamphetamine, caffeine, mescaline, methamphetamine, amphetamine derivatives, dextromethorphan, loperamide, phenylcyclohexyl piperidine, stimulants, steroids, cannabinoids, cathinones, lysergic acid diethylamide, gambling, sex, inhalants, sedatives, hypnotics, tobacco, anxiolytics, hallucinogens, food, a behavior, a repetitive behavior, a destructive habit, or a combination thereof.

5. The method of any one of claims 1-4, wherein the composition is administered topical-lingually.

6. The method of claim 5, wherein the composition is formulated as an oral dissolving tablet, a lozenge, a film, a spray, a semisolid, a particulate, or in a lipid-based carrier.

7. The method of any one of claims 1-4, wherein the composition is administered intranasally.

8. The method of claim 7, wherein the composition is formulated as a spray, a semisolid, a particulate, or in a lipid-based carrier.

9. The method of any one of claims 1-4, wherein the composition is administered topically to the gastrointestinal (GI) tract.

10. The method of claim 9, wherein the composition is formulated as a GI patch.

11. The method of any one of claims 1-4, wherein the composition is administered intrarectally.

12. The method of claim 11, wherein the composition is formulated as a suppository.

13. The method of any one of claims 1-12, wherein the dose of the agent is from 1 ng to 20 mg per 100 kg body weight.

14. The method of claim 13, wherein the dose of the agent is from 1 ng to 1 μg per 100 kg body weight.

15. The method of claim 13, wherein the dose of the agent is from 1 μg to 1 mg per 100 kg body weight.

16. The method of claim 13, wherein the dose of the agent is from 1 mg to 20 mg per 100 kg body weight.

17. The method of any one of claims 1-16, wherein the composition comprises PYY.

18. The method of claim 17, wherein the PYY is PYY(3-36).

19. The method of any one of claims 1-16, wherein the composition comprises GLP-1.

20. The method of any one of claims 1-16, wherein the composition comprises leptin.

21. The method of any one of claims 1-16, wherein the composition comprises amylin.

22. The method of any one of claims 1-16, wherein the composition comprises insulin.

23. The method of any one of claims 1-16, wherein the composition comprises calcitonin.

24. The method of any one of claims 1-23, wherein the neural receptor comprises a Y receptor (YR), a Y1 receptor (Y1R), a Y2 receptor (Y2R), a Y4 receptor (Y4R), a Y5 receptor (Y5R), a G-protein coupled receptor (GPRCR), a leptin receptor (LEPR), a GLP-1 receptor (GLP-1R), an insulin receptor (INSR), a glucose-dependent insulinotropic polypeptide receptor (GIPR), a ghrelin receptor (GHS-R), a cholecystokinin A receptor (CCKAR), a cholecystokinin B receptor (CCKBR), or a calcitonin receptor (CALCR).

25. A composition comprising an agent selected from PYY, GLP-1, leptin, amylin, and calcitonin, or an analog, variant, or biologically active fragment thereof, wherein the composition is formulated for intranasal administration and activates a neural receptor of a subject without substantially changing the concentration of the agent in the blood of the subject.

26. The composition of claim 25, wherein the composition is formulated as a spray, a semisolid, a particulate, or in a lipid-based carrier.

27. A composition comprising an agent selected from PYY, GLP-1, leptin, amylin, insulin, and calcitonin, or an analog, variant, or biologically active fragment thereof, wherein the composition is formulated for topical administration to the GI tract and activates a neural receptor of a subject without substantially changing the concentration of the agent in the blood of the subject.

28. The composition of claim 27, wherein the composition is formulated as a GI patch.

29. A composition comprising an agent selected from PYY, GLP-1, leptin, amylin, insulin, and calcitonin, or an analog, variant, or biologically active fragment thereof, wherein the composition is formulated for intrarectal administration and activates a neural receptor of a subject without substantially changing the concentration of the agent in the blood of the subject.

30. The composition of claim 29, wherein the composition is formulated as a suppository.

31. The composition of any one of claims 25-30, wherein the dose of the agent is from 1 ng to 20 mg.

32. The composition of claim 29, wherein the dose of the agent is from 1 ng to 1 μg.

33. The composition of claim 29, wherein the dose of the agent is from 1 μg to 1 mg.

34. The composition of claim 29, wherein the dose of the agent is from 1 mg to 20 mg.

35. The composition of any one of claims 25-34, wherein the composition comprises PYY.

36. The composition of claim 35, wherein the PYY is PYY(3-36).

37. The composition of any one of claims 25-34, wherein the composition comprises GLP-1.

38. The composition of any one of claims 25-34, wherein the composition comprises leptin.

39. The composition of any one of claims 25-34, wherein the composition comprises amylin.

40. The composition of any one of claims 25-34, wherein the composition comprises calcitonin.

41. The composition of any one of claims 27-34, wherein the composition comprises insulin.

42. The composition of any one of claims 25-41, wherein the neural receptor comprises a YR, a Y1R, a Y2R, a Y4R, a Y5R, a GPCR, a LEPR, a GLP-1R, an INSR, a GIPR, a GHS-R, a CCKAR, a CCKBR, or a CALCR.
